(19) United States
(12) Patent Application Publication
(10) Pub. No.: US 2024/0207822 A1
(43) Pub. Date: Jun. 27, 2024

(54) MULTIFUNCTIONAL HYBRID CATALYST WITH NIOBIUM AND TIN SUPPORTED ON HEXAGONAL MESOPOROUS SILICA, SYNTHESIS PROCESS OF SAID CATALYST AND PROCESS FOR OBTAINING BIODEGRADABLE LUBRICATING BASE OILS USING SAID CATALYST

(71) Applicants: PETROLEO BRASILEIRO S.A. - PETROBRAS, Rio De Janeiro (BR); UNIVERSIDADE FEDERAL DO CEARA

(72) Inventors: Solange Assunção QUINTELLA, Fortaleza (BR); Jose Andre Cavalcanti Da Silva, Rio de Janeiro (BR); Francisco Murilo Tavares De Luna, Fortaleza (BR); Charles Lima Bessa Assuncao, Rio de Janeiro (BR); Maria Thiciane Almeida Costa, Fortaleza (BR); Celio Loreiro Cavalcante, JR., Fortaleza (BR); Maria Imaculada G. Silveira Teixeira, Fortaleza (BR)

(21) Appl. No.: 18/545,922

(22) Filed: Dec. 19, 2023

(30) Foreign Application Priority Data
Dec. 21, 2022 (BR) BR 1020220262454

(51) Int. Cl. B01J 23/20; B01J 21/08; B01J 35/30; B01J 35/45; B01J 35/61; B01J 35/63; B01J 35/64; B01J 37/00; B01J 37/02; B01J 37/03; B01J 37/04; B01J 37/08; C07C 67/26; C10M 105/34; C10N 20/00; C10N 20/02

(52) U.S. Cl. CPC B01J 23/20; B01J 21/08; B01J 35/394; B01J 35/45; B01J 35/617; B01J 35/635; B01J 35/638; B01J 35/647; B01J 37/009; B01J 37/0236; B01J 37/038; B01J 37/04; B01J 37/088; C07C 67/26; C10M 105/34; C10M 2207/2815; C10N 2020/02; C10N 2020/081

(57) ABSTRACT

The present invention relates to a multifunctional hybrid catalyst with niobium and tin supported on hexagonal mesoporous silicas (HMS_{Nb—Sn}), synthesis process thereof through isomorphic substitutions and the process for obtaining biodegradable lubricating base oils using said catalyst.

### FIELD OF THE INVENTION

The present invention relates to a multifunctional hybrid catalyst with niobium and tin supported on hexagonal mesoporous silicas (HMSNb—Sn), synthesis process thereof and process for obtaining biodegradable lubricating base oils using said catalyst.

The present invention has application in the area of supply and biofuels as well as renewable and sustainable energy sources.

### BACKGROUND OF THE INVENTION

Non-renewable fossil raw materials are still the main source for the production of energy, fuels and chemicals. However, fossil raw material resources are gradually decreasing as the rate of consumption increases exponentially to meet energy needs derived from rapid population growth. Furthermore, fossil raw materials have the disadvantage of emitting greenhouse gases such as CO₂, NOₓ, among others, which are one of the main factors for global warming.

In this way, the increase in the energy consumption rate and abnormal climate changes, due to rapid population growth and the emission of environmental pollutants, respectively, have led to the search for new, sustainable, renewable and safer energy sources.

Base oils, which are the main constituents of lubricants, predominantly come from non-renewable fossil raw materials (petroleum). In this way, biolubricants are an excellent alternative in the development of renewable and sustainable energy sources because they do not depend on fossil raw materials and because they have advantageous characteristics, such as: high biodegradability, low sulfur content, excellent lubricity, high flash point and low eco toxicity.

Biodegradable base oils are generally obtained from renewable sources (vegetable oils and animal fats) and can be formulated for various applications, such as agricultural equipment and maritime transport, which present risks to the environment by releasing pollutant gases. In this sense, it is noteworthy that several routes using triglyceride transesterification and fatty acid esterification processes have been developed to obtain biodegradable base oils.

In addition to the type of raw material and the synthetic route used, the catalysts used in the reaction steps to obtain biodegradable base oils are essential to enable the formation of these desired products and make the entire process viable. Homogeneous catalysts, for example, are in the same phase as the reactants, thus providing better interaction between these components and resulting in better reaction yield. However, one of the limitations presented by these catalysts is the difficulty of recovery after the reaction, being a waste material and, possibly, another environmental polluting agent. Heterogeneous catalysts are found in different phases from the reactant. In this way, the reactants and products, which are in the liquid or gaseous phases, remain attached to a solid surface (the catalyst) through covalent interactions or adsorption, making it possible to efficiently separate the product formed from the catalyst. Consequently, heterogeneous catalysts can be synthesized for greater specificity, in addition to the possibility of efficient reuse, thus generating high quality products and facilitating subsequent purification processes.

Multifunctional hybrid catalysts have attracted attention due to the union of different active phases (different active centers), which results in high activity in chemical reactions of different nature. Their synthesis can occur from a variety of modifications made to the support structures and the incorporation of active phases.

Research related to the processes for obtaining biodegradable base oils has been extensive, and the catalytic systems typically used in the synthesis reactions of these products are based on metal oxide catalysts supported on SiO₂, SiO₂/Al₂O₃ and clay minerals. However, the methodology used in the synthesis of these catalysts is related to the impregnation of these oxides and, subsequently, the reduction of these materials at high temperatures as well as in a reducing atmosphere, making the processes more complex and the materials more susceptible to leaching.

In the present invention, hybrid catalysts were specifically developed with Niobium (Nb) and Tin (Sn) metals supported on Hexagonal Mesoporous Silicas (HMS) for applications in reactions to obtain biodegradable base oils. These materials can be modified by replacing silicon atoms with Niobium (Nb) and Tin (Sn) metals in the silicate structures. The grouping of metallic nanoparticles in the mesoporous network expands its applications, and causes it to have high catalytic activity, depending on the procedures for incorporating the active phases. In this way, the inorganic network modified with these nanoparticles through the isomorphic replacement of silicon by metallic nanoparticles promotes good resistance due to the improvement of its structural properties, such as increased acidity, improving its selectivity and increasing its catalytic activity.

With regard to the disclosed state of the art, it is noteworthy that although the document IN 1632 DEL teaches a process for the preparation of active hexagonal mesoporous silica catalyst, it does not disclose obtaining multifunctional catalysts with niobium and tin supported on Hexagonal Mesoporous Silicas (HMS_{Nb—Sn}), as disclosed in the present invention.

Although document US 2015353857 discloses a route for obtaining lubricants from vegetable oils involving epoxidation and esterification steps, the catalysts used are different, consequently, their obtained results.

Therefore, no document from the state of the art teaches the combination, disclosed in the present invention, of mesoporous materials with high surface area and different stable active phases, which favor the diffusion and reaction of reactant and product molecules, thus playing a fundamental role for high catalytic performance.

### SUMMARY OF THE INVENTION

The present invention relates to multifunctional hybrid catalysts containing niobium and tin synthesized by isomorphic substitutions and applied in reactions in the process of obtaining biodegradable base oils.

### BRIEF DESCRIPTION OF THE FIGURES

The present invention will be described below, with reference to the attached figures which, in a schematic way and not limiting the inventive scope, represent examples of its implementation.

FIG. 1 illustrates the synthesis scheme of the HMS_{Nb—Sn} catalyst.

FIG. 2 illustrates a scheme for intensifying the process of obtaining biodegradable base oils.

FIG. 3 illustrates the reaction scheme of the process for obtaining biodegradable base oils with a multifunctional hybrid catalyst.

FIG. 4 illustrates a graph relating to nitrogen adsorption/desorption isotherms at 77 K of the HMS_{Nb—Sn} catalyst.

FIG. 5 illustrates a graph relating to Programmed Temperature Reduction (TPR) with the catalyst (HMS_{Nb—Sn}) and support (HMS) samples.

### DETAILED DESCRIPTION OF THE INVENTION

The present invention discloses a multifunctional hybrid catalyst with niobium and tin supported on HMS_{Nb—Sn}, synthesis process thereof through isomorphic substitutions and the process for obtaining biodegradable lubricating base oils using said catalyst.

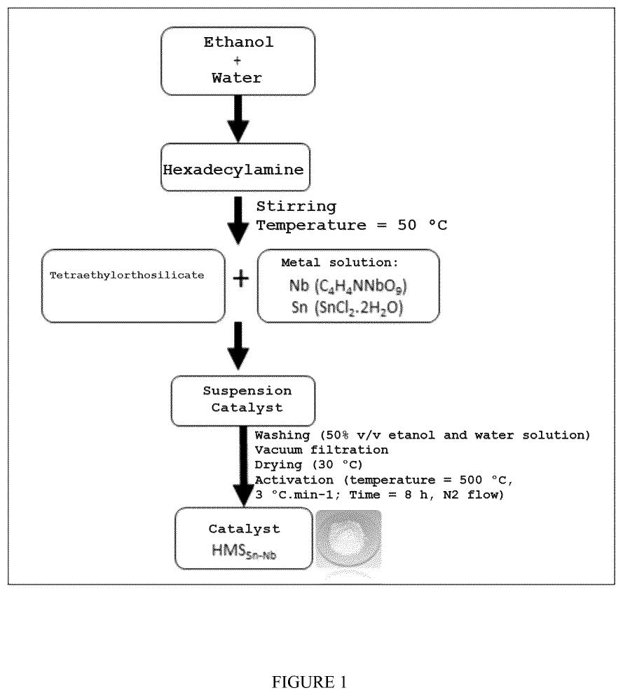

As can be seen in FIG. 1, the hybrid catalysts of the present invention were obtained through the synthesis process, where they were modified by replacing silicon atoms with Nb and Sn metals in the silicate structures. In this way, it was noted that the inorganic network modified through the isomorphic replacement of silicon by metallic nanoparticles promoted good resistance due to the improvement of its structural properties, such as increased acidity, thus improving its selectivity and increasing its catalytic activity, depending on the procedures for incorporating active phases. The support without the incorporation of active phases has practically no catalytic activity as it does not present defects in the network, redox and acidity properties that are important for applications in the developed processes. After the modifications, the catalysts became active due to the action of metals (Sn and Nb) incorporated into the materials. Tin, being tetrahedrally coordinated in this type of material, acts as Lewis acid sites, while niobium, which is pentacoordinated, acts as Brönsted-Lowry acid sites. The combined incorporation of these metals modifies the surface of the mesoporous support more effectively than the incorporation of a monoheteroatom. The synergy of this combination was verified through the catalytic efficiency in simultaneous reactions of esterification and opening of the oxirane rings.

The synthesized multifunctional hybrid catalysts (HMS_{Nb—Sn}) of the present invention can be applied in the process of obtaining biodegradable base oils from unsaturated fatty acids derived from vegetable oils, using the esterification and deionization reactions in a single reaction step of oxirane ring opening with excellent performance.

Said catalysts as well as the chemical reaction route of the process of obtaining said oils demonstrated to be efficient for obtaining biodegradable lubricating base oils with advantageous physicochemical properties for the formulation of various products, such as: hydraulic fluids, cutting fluids, turbine oils, industrial gear oils and compressor oils.

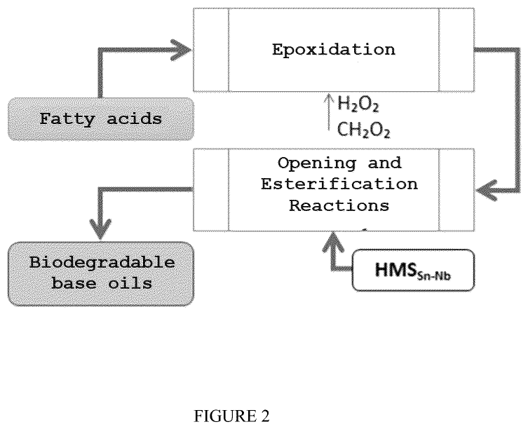

The process for obtaining biodegradable base oils via heterogeneous catalysis using niobium and tin hybrid catalysts supported on hexagonal mesoporous silica demonstrated high catalytic activity in the joint reaction step of esterification and opening of the oxirane rings (FIG. 2). This is due to the fact that mesoporous materials with high surface area and different stable active phases favor the diffusion and reaction of reactant and product molecules, thus playing a fundamental role in high catalytic performance. Therefore, it was observed that hybrid catalysts favored the intensification of the process by helping to reduce reaction steps (simultaneous esterification reactions and opening of the oxirane ring), which has a direct impact on the operational cost and overall efficiency of the process, due to lower number of unit operations, reduction in reaction time, reduction in the amount of reagents, and costs with recovery and purification of products.

As illustrated in FIG. 1, the multifunctional hybrid catalyst with niobium and tin supported on mesoporous hexagonal silicas of the present invention were synthesized as follows.

### 1. Preparation of the HMS_{Nb—Sn} Catalyst: Synthesis Procedures for Niobium and Tin Hybrid Catalysts Supported on HMS

The following materials and their respective amounts as well as methodology were used in the synthesis process of multifunctional hybrid catalyst with niobium and tin supported on hexagonal mesoporous silicas of the present invention:

| Material | Amount | |
|---|---|---|
| Water | 20 to 40 | ml |
| Ethanol | 15 to 35 | ml |
| Hexadecylamine (HAD) | 2 to 5 | ml |
| TEOS | 5 to 25 | ml |
| C₄H₄NNbO₉ | 0.01 to 0.05 | g |
| SnCl₂.2H₂O | 0.05 to 0.20 | g |

As illustrated in the scheme of FIG. 1, an alcoholic solution was prepared by diluting 25 ml of ethanol in 30 ml of distilled water, followed by the addition of 3 ml of Hexadecylamine (HDA), under stirring at 500 rpm, at temperature of 50° C. until homogenization. Subsequently, the solution containing niobium ammonium oxalate (0.03 g) and tin chloride (0.15 g) with Tetraethylorthosilicate (TEOS) (15 ml) was added to said alcoholic solution. Constant magnetic stirring at 500 rpm of the mixture was carried out for 15 min. After stirring, the mixture was kept at rest for 24 h. Washing the material obtained with an ethanol/water solution (50% v/v) and vacuum filtration were carried out after resting. The resulting solid was dried at a temperature of 30° C. and calcined at 500° C. for 8 h, under a N₂ flow (20 ml/min) and a heating rate of 3° C./min, to obtain and activate the HMS_{Sn—Sb} catalyst.

In this way, the synthesized catalyst obtained has the following components and properties, with the percentage values of the main components of the HMS_{Nb—Sn} catalyst illustrated in Table 1 below:

TABLE 1
Final composition of the mass percentage of the components

| | Components | mass % |
|---|---|---|
| Support | HMS | 95 to 99.2 |
| Active phase | Nb | 0.5 to 3.0 |
| | Sn | 0.3 to 2.0 |

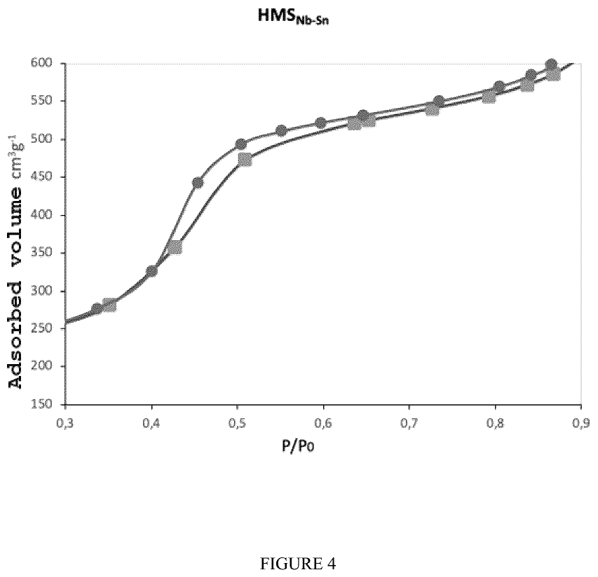

The nitrogen adsorption/desorption isotherms at 77 K for a representative sample of the HMS_{Nb—Sn} catalyst are illustrated in FIG. 4. They present type IV behavior, which are characteristics of mesoporous materials. Using the data obtained from the isotherms, it was possible to calculate the surface area (BET), the total pore volume and the average pore diameter, that is, the textural properties of the catalysts. Table 2 shows the values obtained in a representative range for the prepared materials. The high surface area and high pore volume favored the expansion of the availability of metallic active sites, and the average pore sizes were adequate to improve the accessibility of reactants to active sites and the diffusion of products.

TABLE 2
Textural properties of HMS_{Nb—Sn} catalysts

| Textural properties | HMS_{Nb—Sn} |
|---|---|
| Surface area BET (m²/g) | 800 to 900 |
| Total pore volume (cm³/g) | 0.98 to 1.3 |
| Average pore diameter (Å) | 52 to 56 |

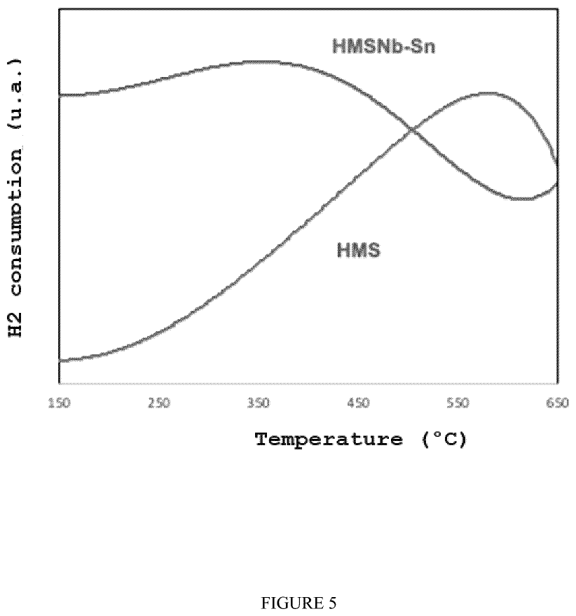

FIG. 5 shows the curves for the Programmed Temperature Reduction (TPR) experiments for the catalyst (HMS_{Nb—Sn}) and catalytic support (HMS) samples. It is observed that the reduction range of the precursor metal varies from 350° C. to 550° C., which are characteristics of reduction of the metallic species of Nb and Sn, verifying the incorporation of the active phases in the catalyst (HMS_{Nb—Sn}).

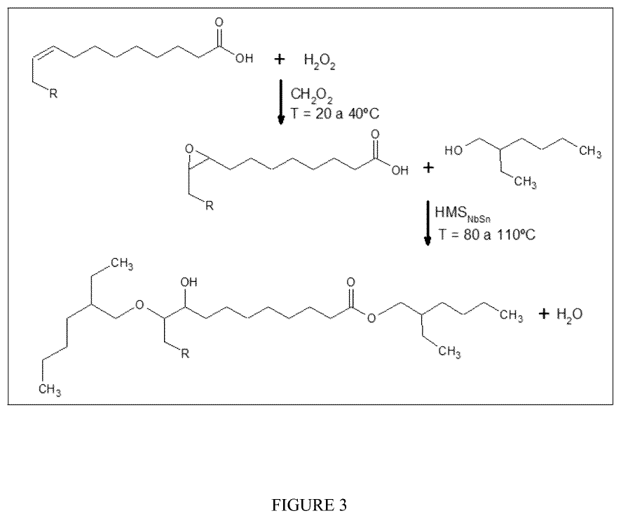

The catalytic activities of the materials were monitored, by Vibrational Spectroscopy in the infrared region (FTIR) and by Nuclear Magnetic Resonance (¹H and ¹³C), in the esterification and opening reactions of the oxirane rings, initially in isolation, using the 2-ethylhexanol alcohol as an esterifier or as a nucleophilic agent. It was possible to observe that the HMS_{Nb—Sn} multifunctional hybrid catalysts provided high conversion and selectivity for isolated reactions and in a single step (FIGS. 2 and 3). The combination of niobium and tin metals, supported on mesoporous material (HMS), favored the simultaneous reactions of esterification and opening of the oxirane rings due to the porous structure of the materials and the action of the metallic active sites that promoted the nucleophilic attack reactions of the oxirane groups and formation of esters, thus increasing selectivity to the desired products (biodegradable esters).

2. Process for Obtaining Biodegradable Base Oils from the Synthesized Catalyst of the Present Invention

As revealed in FIGS. 2 and 3, the process for obtaining biodegradable base oils from the synthesized catalyst of the present invention comprises the following methodology (steps and reaction/experimental conditions):

### 2.1. Epoxidation Reaction

The following experimental conditions were used in the epoxidation reaction:
Temperature: 20 to 40° C.;
Stirring rotation: 600 to 800 rpm;
Molar ratio of fatty acid/hydrogen peroxide: 1:2 to 1:6;
Molar ratio of fatty acid/formic acid: 1:1 to 1:2;
Molar ratio of hydrogen peroxide, formic acid and fatty acids: 4 to 8:1 to 2:1 to 2;
Reaction time: 12 to 30 hours;
Reflux reaction system in thermostatic bath with temperature control.
Reaction solvents (hexane, cyclohexane or toluene): 150 to 400 ml.
Amount ranges: 20 to 40 g of fatty acids, 2.5 to 4.5 ml of formic acid and 15 to 25 ml of hydrogen peroxide.
Purification process: washing with saturated aqueous NaHCO3 solution containing 5% by mass of NaHCO3 until pH is adjusted to between 6 to 8 and drying with Na2SO4;
Vacuum filtration system;
Distillation in Kugelrohr equipment at 110° C. for 60 minutes.

FIG. 2 shows an overview of the process for obtaining biodegradable base oils. Regarding the epoxidation reaction, this step was carried out in a batch reactor with a homogeneous catalyst, to which 30 g of fatty acids were added. To avoid parallel reactions and premature opening of the oxirane ring with water, 150 mL of toluene (solvent) were added. The reactor was coupled to a reflux system and with magnetic stirring at 800 rpm. The molar ratio used in this reaction was 4:1:1 of hydrogen peroxide, formic acid and unsaturated fatty acids, respectively, indicating a stoichiometric excess of 300% of hydrogen peroxide. The addition of 20 ml of hydrogen peroxide to the reaction medium was carried out slowly, lasting approximately 1.5 h, to avoid excessive heating of the medium, as it is an exothermic reaction. After completing the addition, the reaction was carried out for 24 h at room temperature (approximately 25° C.) to ensure complete epoxidation of the products. The epoxidized product was washed in a separating funnel using distilled water and 5% of sodium bicarbonate solution. Several washes were carried out until the pH of the water was close to 7. Then, anhydrous sodium sulfate was added to the epoxidation product and left to rest for one hour at room temperature to remove the water from the ester phase. Finally, the sample was distilled in Kugelrohr at 110° C. for 1 hour to recover excess solvents.

The aforementioned epoxidation reaction is aimed at unsaturated fatty acids, such as those making up soybean, castor, cotton, canola, sesame, pequí oils, among others. Obviously, some vegetable oils have lower amounts of unsaturated fatty acids, for example, palm and babassu oil, thus generating products, obtained by the process of the present invention, with different properties.

### 2.2. Esterification Reactions and Oxirane Ring Opening

The following experimental conditions were used in the esterification reaction and the oxirane ring opening:
Temperature: 80 to 110° C.;
Stirring rotation: 700 to 1100 rpm;
Catalyst/epoxidized product mass ratio: 0.03 to 0.06 g/g;
Molar ratio epoxidized product/2-ethylhexanol: 1:2 to 1:6;
Reaction time: 5 to 8 hours;
Inert nitrogen atmosphere with a flow of 1 to 5 mL/min;
Reflux reaction system with thermostatic bath.
Ranges in mass values (g): 35 to 60 g of the epoxidized product; 45 to 70 g of 2-ethylhexanol: 1.5 to 3.0 of the HMSNb—Sn catalyst;
Vacuum filtration system for catalyst separation;
Distillation in Kugelrohr equipment at 110 to 125° C. for 60 minutes.

As can be seen in FIGS. 2 and 3, the epoxidized product was simultaneously subjected to oxirane ring opening and esterification using a batch reactor with heterogeneous catalyst (HMS_{Nb—Sn}). In this way, 40 g of the epoxidized product was added and 52.4 g of 2-ethylhexanol with a molar ratio of 1:3 (epoxidized product/2-ethylhexanol). The amount of catalyst used was 5% in relation to the mass of epoxidized product (2.0 g). The reaction was subjected to magnetic stirring at a constant rotational speed of 800 rpm, in an inert nitrogen atmosphere with a flow rate of 1.5 mL/min. The reaction temperature was controlled by remaining at 85° C. for 6 hours. Finishing the reaction, the catalyst was removed by vacuum filtration and the product resulting from the reaction (biolubricant) was distilled using Kugelrohr equipment at 125° C. for 1 h to remove excess alcohol.

As can be seen in FIG. 3, the alcohol 2-ethylhexanol reacts with the epoxidized acid, in the presence of the HMS_{Nb—Sn} catalyst. In this case, the two esterification reactions and the opening of the oxirane rings occur simultaneously to form the final product. It is possible to observe, in FIG. 3, the representation of the simultaneous reactions (intensification of the process) of opening and esterification, when the alcohol (2-ethylhexanol) is being reacted in two groups of the same molecule of epoxidized unsaturated fatty acids.

The final product obtained was monitored by Nuclear Magnetic Resonance (¹H and ¹³C), by spectroscopy in the Fourier transform infrared region (FTIR), by various physicochemical analyzes (specific mass, viscosity, viscosity index, fluidity and acidity) and oxidative stability. Through this monitoring, it was possible to observe that in addition to generating products with desirable physicochemical properties and oxidative stability for use as lubricating base oils, the products also showed high biodegradability (half-life of 23 days), measured using the ASTM D7373 method.

### Catalyst Performance Tests

Catalyst performance tests were carried out in the esterification and oxirane ring opening reactions, using several reuse cycles, and compared with the catalytic activity of another commercial catalyst (Amberlyst 15), with similar results. The advantage of HMS_{Nb—Sn} is reusability and the ability to withstand temperatures above 120° C., while Amberlyst 15 has a limited temperature of use (sulfonic resin), which decomposes and loses its catalytic activity. The structure of the hexagonal mesoporous silica developed and the stability of the incorporated metals favored the maintenance of catalytic activity after several cycles of reuse.

Therefore, the laboratory studies of the present invention, mentioned above, showed that the synthesized catalysts, according to the scheme illustrated in FIG. 1, showed high catalytic activity in the esterification reactions and opening of epoxide groups. The main variables obtained in response to the use of the catalyst in the process for obtaining biodegradable base oils are:
Reagent conversions: >90% (esterification) and >95% (opening);
Viscosity of products from 6 to 12 cSt (100° C.)(ASTM D445);
Specific mass at 20° C. of 0.89 to 0.96 g/cm³ (ASTM D1298);
Total acidity value of 0.5 to 2.0 mg KOH/g (ASTM D664);
Viscosity index (VI) from 100 to 160 (ASTM D2270);
Pour point from −30° C. to −42° C. (ASTM D97);
Oxidative stability from 10 h to 20 h (Rancimat Method at 110° C., under air flow of 1 L/h); and
Biodegradability from 20 days to 30 days (half-life measured by the ASTM D7373 method).

The products obtained, from the process using a shorter synthesis route and with multifunctional niobium and tin catalysts, had excellent physicochemical characteristics that favor their use for formulating biodegradable lubricants. The products obtained were characterized through measurements of viscosity, specific mass, fluidity, acidity, oxidative stability, biodegradability, and chemical and compositional analyzes such as vibrational spectroscopy in the infrared region (FTIR) and Nuclear Magnetic Resonance (¹H and ¹³C). The excellent catalytic activity of HMS_{Nb—Sn} in the joint reactions of esterification and opening of the oxirane rings (conversions above 90%) favor the viability of producing biodegradable base oils with the reduction of reaction steps that directly impact operational and product costs.

1. MULTIFUNCTIONAL HYBRID CATALYST, characterized by comprising metallic nanoparticles supported on a mesoporous network.

2. CATALYST, according to claim 1, characterized in that the metallic nanoparticles comprise niobium (Nb) and tin (Sn).

3. CATALYST, according to claim 1 or 2, characterized in that Nb and Sn comprise the different active phases of the catalyst.

4. CATALYST, according to claim 1, characterized in that the mesoporous network comprises hexagonal mesoporous silica (HMS).

5. CATALYST, according to any one of claims 1 to 4, characterized by comprising mass % of 95 to 99.2% of HMS as support and 0.5 to 3% of Nb and 0.3 to 2% of Sn as different active phases.

6. CATALYST, according to any one of claims 1 to 5, characterized by having the following properties: surface area of 800 to 900 m²/g, total pore volume of 0.98 to 1.3 cm³/g and average pore diameters of 52 to 56 Å.

7. SYNTHESIS PROCESS FOR OBTAINING THE MULTIFUNCTIONAL HYBRID CATALYST as defined in claim 1, characterized by comprising the following steps:
preparing an alcoholic solution by diluting ethanol in distilled water;
adding hexadecylamine (HDA) to the alcoholic solution, under stirring at 500 rpm, at a temperature of 50° C. until homogenization;
then, adding a metal solution containing niobium ammonium oxalate and tin chloride with tetraethylorthosilicate (TEOS) and magnetically stirring the mixture at 500 rpm for 15 min;
resting the suspension obtained for 24 h, after stirring;
washing the material obtained with a 50% v/v ethanol/water solution;
filtering under vacuum;
drying the solid at a temperature of 30° C.; and
calcining up to 500° C. for 8 h, under N₂ flow (20 ml/min), at a heating rate of 3° C./min.

8. PROCESS, according to claim 7, characterized in that the replacement of silicon atoms by Nb and Tin Sn metals in the silicate structures occurs during the step of adding the metal solution with TEOS under magnetic stirring.

9. PROCESS FOR OBTAINING BIODEGRADABLE BASE OILS USING THE MULTIFUNCTIONAL HYBRID CATALYST as defined in claim 1, characterized by comprising the following steps:
subjecting unsaturated fatty acids to the epoxidation reaction in the presence of toluene, formic acid and hydrogen peroxide, in a stoichiometric excess of hydrogen peroxide; and
subjecting the epoxidized acid, simultaneously, to the esterification reaction and opening the oxirane ring through the addition of 2-ethylhexanol, in stoichiometric excess in the presence of the HMS_{Nb—Sn} catalyst.

10. PROCESS, according to claim 9, characterized in that the amount of catalyst used was 5% in relation to the mass of epoxidized product.

11. PROCESS, according to claim 9 or 10, characterized in that the epoxidation reaction was carried out in a batch reactor with a 500 ml of heterogeneous catalyst (HMS_{Nb—Sn}) coupled to a reflux system and with constant magnetic stirring of 800 rpm with a stoichiometric excess of 300% of hydrogen peroxide, in which the epoxidation reaction was carried out for 24 h at room temperature until the products were completely epoxidized.

12. PROCESS, according to claim 11, characterized in that the epoxidized product obtained was subjected to the following steps:
washing in a decantation funnel, using distilled water and a 5% of sodium bicarbonate solution, until the pH of the water was close to 7;
adding anhydrous sodium sulfate to the epoxidation product left for one hour at room temperature, separating the water from the ester phase; and
distilling the sample in Kugelrohr at 60° C. for 1 hour to recover excess solvents.

13. PROCESS, according to claim 9, characterized in that the simultaneous reaction of opening the oxirane ring and esterification was carried out in a batch reactor with a heterogeneous catalyst (HMS_{Nb—Sn}) of 500 ml under magnetic stirring at rotational speed of 800 rpm, in an inert nitrogen atmosphere with a flow of 1.5 mL/min, with the reaction temperature controlled, remaining at 85° C. for 6 hours.

14. PROCESS, according to claim 13, characterized in that 90% or more of the reactants in the esterification and 95% or more of the reactants in the ring opening have been converted.

15. PROCESS, according to any one of claims 9 to 13, characterized in that the catalyst used was removed by vacuum filtration and the product resulting from the reaction was distilled using Kugelrohr equipment at 125° C. for 1 h to remove excess of alcohol.

16. PROCESS, according to claim 1, characterized by obtaining the basic lubricating oil with the following characteristics:
Viscosity of products from 6 to 12 cSt (100° C.) (ASTM D445);
Specific mass at 20° C. of 0.89 to 0.96 g/cm³ (ASTM D1298);
Total acidity value of 0.5 to 2.0 mg KOH/g (ASTM D664);
Viscosity index (VI) from 100 to 160 (ASTM D2270);
Pour point from −30° C. to −42° C. (ASTM D97);
Oxidative stability from 10 h to 20 h (Rancimat Method at 110° C., under air flow of 1 L/h); and
Biodegradability from 20 days to 30 days (half-life measured by the ASTM D7373 method).
